(19) United States
(12) Patent Application Publication
(10) Pub. No.: US 2020/0359876 A1
(43) Pub. Date: Nov. 19, 2020

(54) SURGICAL DEVICE GRIP

(71) Applicant: ARTHREX, INC., Naples, FL (US)

(72) Inventors: Paul Waters, Santa Barbara, CA (US); Benjamin Hedges, Santa Barbara, CA (US)

(21) Appl. No.: 16/412,942

(22) Filed: May 15, 2019

(51) Int. Cl. A61B 1/00

(52) U.S. Cl. CPC A61B 1/00066; A61B 1/0052; A61B 1/00147

(57) ABSTRACT

A grip for a surgical device, the grip having an inner portion with a first material, a cylindrical opening, and at least one living hinge; an outer portion coupled to the inner portion, the outer portion having a second material different than the first material, and an outer surface; wherein the inner portion is configured so that the cylindrical opening fits around the surgical device and the living hinge is adjustable to permit sliding along the surgical device.

### BACKGROUND

The present invention relates to devices used in endoscopic surgery and, more particularly, to grips for cannulas and endoscopes used in endoscopic surgery.

Endoscopic camera systems provide a surgeon with a view of an area of the body being worked on without the necessity of fully opening up the area, thereby allowing for less invasive surgical procedures. Generally, these devices consist of a camera head coupled to an endoscope and a camera control unit. Recent miniaturization of image sensors has led to the development of very small diameter endoscopes that are also light weight and less bulky than their traditional counterparts. Small image sensors can be placed at a distal tip of an endoscope with a diameter of less than 2 mm. Endoscopes are typically stainless steel tubes that are hard to grip. Often, there is no significant endoscope proximal housing for a surgeon to grasp to manipulate the endoscope. Even when an endoscope is placed in a sheath or cannula the diameter often does not exceed 2.5 mm. Because of their small size, it is difficult to manipulate and hold the sheath or cannula firmly during insertion.

It would be desirable to have a system and method for better gripping the endoscopes and cannulas used in endoscopic surgery that improves upon the prior art.

### SUMMARY

This invention is a device that slips over an endoscope or cannula to provide greater friction and a larger diameter for more effective and comfortable manual support and control.

According to an embodiment, the present invention is directed to a grip for a surgical device, the grip comprising: an inner portion further comprising: a first material; a cylindrical opening; and at least one living hinge. An outer portion is coupled to the inner portion, the outer portion further comprising: a second material different than the first material; and an outer surface. The inner portion is configured so that the cylindrical opening fits around the surgical device and the living hinge is adjustable to permit sliding along the surgical device.

In an embodiment, the outer portion further comprises a first half of an annular tube and a second half of an annular tube; wherein the first half and the second half are coupled to opposite sides of the inner portion. The first material may be rubber and the second material may be plastic. Optionally, the outer portion has a diameter of between about 4 mm and about 8 mm; and more preferably between about 5 mm and about 7 mm. The outer surface may have at least one of an anti-slip texture and a plurality of dimples.

In an embodiment, the inner portion comprises two living hinges positioned on opposite sides of the cylindrical opening. Additionally, the cylindrical opening may be configured to fit around at least one of a cannula and an endoscope shaft. Optionally, the cylindrical opening has a resting diameter of less than about 2 mm.

The present invention, according to an embodiment is also directed to a method of controlling an endoscope. The method comprises the steps of: providing an endoscope for insertion into a body cavity, the endoscope further comprising a shaft; providing a cannula configured to fit around the endoscope shaft; and providing a grip. The grip has: an inner portion further comprising: a first material; a cylindrical opening configured to fit around the cannula; and at least one living hinge adjustable to permit sliding of the grip along the cannula. The grip also has an outer portion coupled to the inner portion, the outer portion further comprising: a second material different than the first material; and an outer surface. The method further comprises the steps of opening the at least one living hinge; placing the cannula through the cylindrical opening of the grip; closing the at least one living hinge; and controlling the cannula and endoscope with the grip.

In an additional embodiment, the outer portion further comprises a first half of an annular tube and a second half of an annular tube, the first and second halves being coupled to opposite sides of the inner portion; and the inner portion further comprises two living hinges positioned on opposite sides of the cylindrical opening. The step of opening the at least one living hinge further comprising pushing both living hinges toward each other to expand the cylindrical opening.

In an additional embodiment, the present invention is directed to a grip for a surgical device, the grip comprising: an inner portion further comprising: a first material; a cylindrical opening; and two living hinges on opposite sides of the cylindrical opening. The grip also has an outer portion coupled to the inner portion, the outer portion further comprising: a second material different than the first material; a first half of an annular tube; and a second half of an annular tube; wherein the first half and the second half are coupled to opposite sides of the inner portion; the cylindrical opening is configured to fit around the surgical device; and the living hinges are adjustable to permit sliding along the surgical device.

Optionally, the first material is rubber and the second material is plastic. The outer portion may have a diameter of between about 4 mm and about 8 mm. Additionally, the outer portion may have a diameter of between about 5 mm and about 7 mm. Optionally, the outer surface has an anti-slip texture or a plurality of dimples. Additionally, the cylindrical opening may be configured to fit around at least one of a cannula and an endoscope shaft. Optionally, the cylindrical opening has a resting diameter of less than about 2 mm.

These and other features are described below.

### BRIEF DESCRIPTION OF THE DRAWINGS

The features, aspects and advantages of the present invention will become better understood with regard to the following description, appended claims and accompanying figures wherein:

FIG. 1 is a schematic diagram of a camera system according to an embodiment of the present invention;

FIG. 2 is a perspective view of an endoscope, cannula and grip system according to an embodiment of the present invention;

FIG. 3 is a perspective elevational view of a grip according to an embodiment of the present invention; and

FIG. 4 is a perspective view of a grip according another embodiment of the present invention.

### DETAILED DESCRIPTION

In the following description of the preferred embodiments, reference is made to the accompanying drawings which show by way of illustration specific embodiments in which the invention may be practiced. Wherever possible, the same reference numbers will be used throughout the drawings to refer to the same or like parts. It is to be understood that other embodiments may be utilized and structural and functional changes may be made without departing from the scope of the present invention.

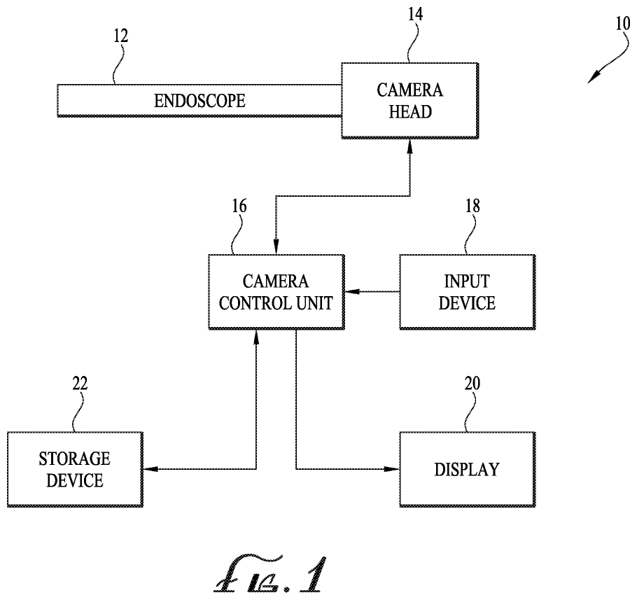

With reference to FIG. 1, an endoscopic camera system 10 according to an embodiment of the present invention has at least one endoscope 12 connectable to a camera head 14. The camera head 14 is connectable to a camera control unit 16 (“CCU”). The camera head 14 and camera control unit 16 may be connected via wire or wirelessly. The camera control unit 16 is also connectable to at least one input device 18 such as a mouse, keyboard, touchpad, or touchscreen monitor. Additionally, the camera control unit is connectable to a display 20 and a storage device 22, such as an external hard drive or server.

The camera head 14 has an imaging device and may also have an illumination system. The camera head 14 may also have memory for storing camera data, camera control unit processing data or other information. The camera head 14 may also have a user input means such as buttons to control aspects of image capture.

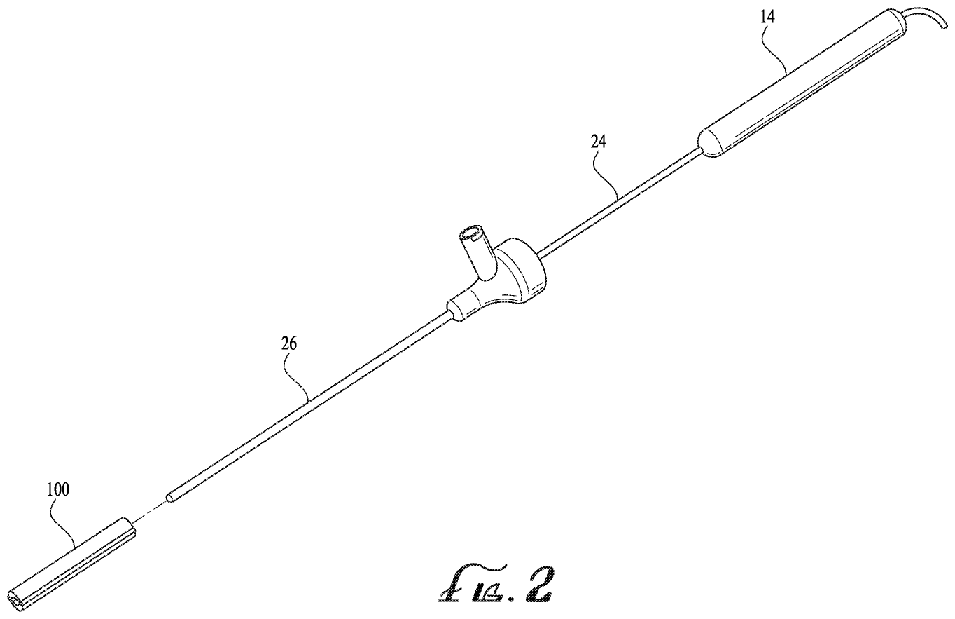

With reference to FIG. 2 the endoscope 12 has a shaft 24 with a diameter of less than about 20 mm, preferably less than about 2.0 mm and more preferably less than about 1.9 mm. The endoscope shaft is less than about 450 mm long. In order to protect the endoscope and aid in insertion in a patient, the endoscope shaft 24 may be inserted into a cannula 26. Typically, the cannula 26 has an outer diameter of less than about 20 mm and preferably less than about 2.25 mm, and a length of less than about 450 mm. A grip 100 according to an embodiment is shown at the lower left of FIG. 2. The grip 100 has a diameter of between about 3 mm and about 25 mm. The grip 100 has a length between about 20 mm and about 130 mm. The grip 100, cannula 26, and endoscope 12 are shown separated along their axes.

In use according to the embodiment shown in FIG. 2, the endoscope shaft 24 is inserted into the cannula 26 and the grip 100 is slipped over the cannula. The position of the grip 100 on the cannula 26 is adjusted by the user depending on the insertion depth of the cannula in the patient. Alternatively, when no cannula 26 is needed, the grip 100 is slipped over the endoscope shaft 24 and adjusted by the user depending on the insertion depth of the endoscope shaft in the patient. Optionally, if the endoscope is longer than the cannula, the grip 100 may be placed over the endoscope shaft 24 to manipulate the endoscope within the cannula.

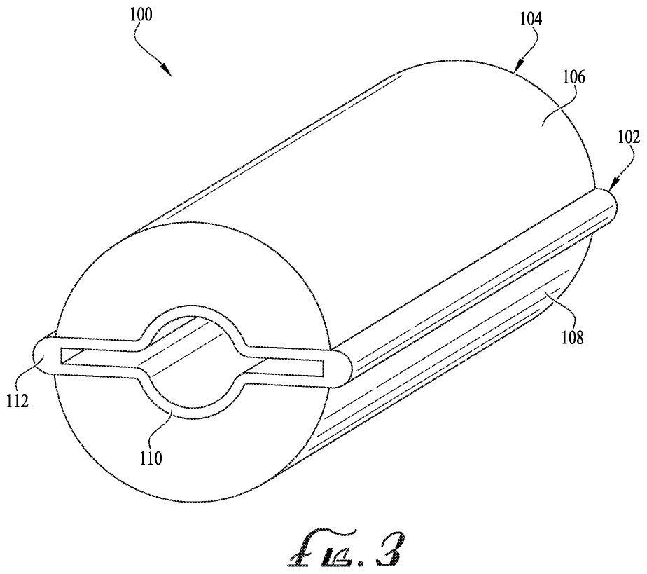

With reference to FIG. 3, the grip 100 has an inner portion 102 and an outer portion 104. In an embodiment, the inner portion 102 and the outer portion 104 are formed from different materials. The outer portion 104 has a first half 106 of an annular tube and a second half 108 of an annular tube. The outer portion 104 may be formed of a hard plastic, such as acrylonitrile butadiene styrene (ABS) plastic such as manufactured by BASF Corporation, Florham Park, N.J. and SABIC, Exton, Pa. The outer portion 104 may also be formed of Polycarbonate-ABS (PC/ABS), polyvinyl chloride (PVC), Polycarbonate, Nylon, Polypropylene or other hard plastic approved for medical applications. In an embodiment, the outer portion has a diameter of between about 4 mm and about 8 mm; and more preferably, between about 5 mm and about 6 mm.

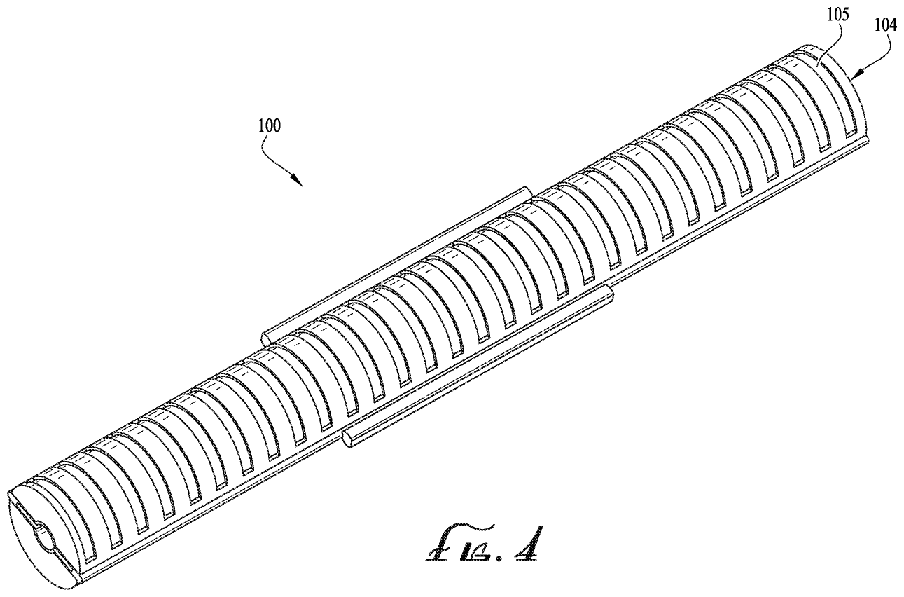

As shown in FIG. 4, the outer portion 104 may have a textured outer surface to improve finger traction. Additionally, the outer portion 104 may have dimples or indentations 105 to improve finger traction. Additionally, the outer portion 104 may include a material soft enough to at least partially conform to the fingers of a user to improve finger traction.

The first half 106 and the second half 108 are joined together by the inner portion 102. The inner portion 102 is configured to form a cylindrical opening 110 and at least one hinge 112. In an embodiment, the hinge 112 is a living hinge. In an embodiment, the inner portion 102 has two living hinges 112 on opposite sides of the cylindrical opening 110. Preferably the cylindrical opening 110 is configured to fit around a cannula and around an endoscope shaft. The inner portion is formed of an elastic and pliable material, such as a rubber elastomeric material with a durometer of 60A and is bonded to the first and second halves 106, 108 of the outer portion 104. The inner portion may be formed of rubber, nitrile, buna, silicone, polyurethane, or combinations thereof.

In an embodiment, the inner portion 102 draws the first and second halves 104, 106 of the outer portion 104 together over the outside of the cannula or the endoscope shaft. In an embodiment, the cylindrical opening 110 has a resting diameter of less than about 8 mm; preferably less than about 2.25 mm; and more preferably less than about 1.9 mm. As will be recognized by those of skill in the art, the resting diameter of the cylindrical opening 110 may be selected based on the outer diameter of the object to be held.

Preferably, the inner portion 102 has a texture that helps secure the inner portion against the outside of the cannula or endoscope shaft once the grip 100 is positioned. The elastic nature of the inner portion 102 allows the inner portion to spread apart for use of the grip on cannulas and endoscope shafts having a range of diameters. In an embodiment, squeezing the living hinges 112 toward each other causes first and second halves 106, 108 of the outer portion 104 to spread apart, thereby releasing the grip from the cannula or endoscope shaft to permit removal or repositioning of the grip 100.

Advantageously, the grip has a diameter that is larger than the diameter of either the cannula or the endoscope shaft, which makes the grip easier for a user to handle and control than directly grasping the cannula or the endoscope shaft. Also advantageously, the combination of different materials for the inner portion 102 and the outer portion 104 allow for a grip 100 that securely engages the cannula or endoscope shaft, but that is also quickly and easily repositionable.

As will be understood by one of the skill in the art, the grip of the present invention can also be used for positioning other hard to grip surgical devices such as, for example, a sheath and a catheter. Additionally, the grip 100 may be disposable.

There is disclosed in the above description and the drawings, a surgical device positioner that fully and effectively overcomes the disadvantages associated with the prior art. However, it will be apparent that variations and modifications of the disclosed embodiments may be made without departing from the principles of the invention. The presentation of the preferred embodiments herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. § 112.

What is claimed is:

1. A grip for a surgical device, the grip comprising:
an inner portion further comprising:
a first material;
a cylindrical opening; and
at least one living hinge;
an outer portion coupled to the inner portion, the outer portion further comprising:
a second material different than the first material; and
an outer surface;
wherein the inner portion is configured so that the cylindrical opening fits around the surgical device and the living hinge is adjustable to permit sliding along the surgical device.

2. The grip of claim 1 wherein the outer portion further comprises a first half of an annular tube and a second half of an annular tube; and
wherein the first half and the second half are coupled to opposite sides of the inner portion.

3. The grip of claim 2 wherein the first material is rubber and the second material is plastic.

4. The grip of claim 2 wherein the outer portion has a diameter of between about 4 mm and about 8 mm.

5. The grip of claim 4 wherein the outer portion has a diameter of between about 5 mm and about 7 mm.

6. The grip of claim 4 wherein the outer surface has at least one of an anti-slip texture and a plurality of indentations.

7. The grip of claim 2 wherein the inner portion comprises two living hinges positioned on opposite sides of the cylindrical opening.

8. The grip of claim 7 wherein the cylindrical opening is configured to fit around at least one of a cannula and an endoscope shaft.

9. The grip of claim 8 wherein the cylindrical opening has a resting diameter of less than about 2 mm.

10. A method of controlling an endoscope comprising the steps of:
providing an endoscope for insertion into a body cavity, the endoscope further comprising a shaft;
providing a cannula configured to fit around the endoscope shaft;
providing a grip comprising:
an inner portion further comprising:
a first material;
a cylindrical opening configured to fit around the cannula; and
at least one living hinge adjustable to permit sliding of the grip along the cannula;
an outer portion coupled to the inner portion, the outer portion further comprising:
a second material different than the first material; and
an outer surface;
opening the at least one living hinge;
placing the cannula through the cylindrical opening of the grip;
closing the at least one living hinge; and
controlling the cannula and endoscope with the grip.

11. The method of claim 10 wherein the outer portion further comprises a first half of an annular tube and a second half of an annular tube, the first and second halves being coupled to opposite sides of the inner portion; the inner portion further comprises two living hinges positioned on opposite sides of the cylindrical opening; and wherein the step of opening the at least one living hinge further comprising pushing both living hinges toward each other to expand the cylindrical opening.

12. A grip for a surgical device, the grip comprising:
an inner portion further comprising:
a first material;
a cylindrical opening; and
two living hinges on opposite sides of the cylindrical opening;
an outer portion coupled to the inner portion, the outer portion further comprising:
a second material different than the first material;
a first half of an annular tube; and
a second half of an annular tube;
wherein the first half and the second half are coupled to opposite sides of the inner portion;
the cylindrical opening is configured to fit around the surgical device; and the living hinges are adjustable to permit sliding along the surgical device.

13. The grip of claim 12 wherein the first material is rubber and the second material is plastic.

14. The grip of claim 12 wherein the outer portion has a diameter of between about 4 mm and about 8 mm.

15. The grip of claim 12 wherein the outer portion has a diameter of between about 5 mm and about 7 mm.

16. The grip of claim 15 wherein the outer surface has at least one of an anti-slip texture and a plurality of indentations.

17. The grip of claim 16 wherein the cylindrical opening is configured to fit around at least one of a cannula and an endoscope shaft.

18. The grip of claim 17 wherein the cylindrical opening has a resting diameter of less than about 2 mm.
